(19) United States
(12) Patent Application Publication
(10) Pub. No.: US 2025/0259754 A1
(43) Pub. Date: Aug. 14, 2025

(54) ARTIFICIALLY INTELLIGENT DIALOG EVALUATION SYSTEM AND ASSOCIATED METHODS

(71) Applicant: ARIZONA BOARD OF REGENTS ON BEHALF OF THE UNIVERSITY OF ARIZONA, Tucson, AZ (US)

(72) Inventors: Allan Hamilton, Tucson, AZ (US); Kyle McLemore, Tucson, AZ (US)

(21) Appl. No.: 19/051,667

(22) Filed: Feb. 12, 2025

Related U.S. Application Data
(60) Provisional application No. 63/552,516, filed on Feb. 12, 2024.

(51) Int. Cl. G16H 80/00; G06F 40/40; G10L 17/02; G16H 10/60

(52) U.S. Cl. CPC G16H 80/00; G06F 40/40; G10L 17/02; G16H 10/60

(57) ABSTRACT

An evaluation device receives captured data of a medical history evaluation interview between a patient and a medical provider. The evaluation device transcribes audio of the captured data into transcribed text and segments the transcribed text into segmented lines according to a speaker of the transcribed text within the audio. The evaluation device generates a plurality of prompts, each prompt corresponding to one of a plurality of interview analysis variables, to control a large language model (LLM) to analyze each of the segmented lines in context of the transcribed text. The evaluation device transmits the plurality of prompts to the LLM and receives LLM responses from the LLM for each of the plurality of prompts. The evaluation device analyzes the LLM responses with respect to a scoring rubric and generates a detail report defining performance of the medical provider during interaction between the patient and the medical provider.

### RELATED APPLICATION

This application claims priority to U.S. Patent Application No. 63/552,516, titled “System and Method for Artificially Intelligent Medical History Evaluation Instrument,” and filed Feb. 12, 2024, which is incorporated herein by reference in its entirety.

### BACKGROUND

Taking a complete and thorough medical history (MHx) during an interview between a healthcare professional and a patient is a crucial skill for the healthcare professional. The interview process involves various elements, including documenting the interview participants, chief complaint (CC), history of present illness (HPI), past medical history (PMHx), and reviewing organ systems (ROS). Beyond documentation, it's essential to establish a trusting relationship between the patient (Pt) and healthcare provider (HCP).

Training in MHx capture requires developing clear communication skills, and avoiding narrow medical vocabulary, technical jargon, and slang. The HCP should ask questions clearly, respectfully, and empathetically, demonstrating active listening to a patients' responses. This approach ensures comprehensive understanding and accurate recording of medical history, facilitating effective diagnosis and treatment. Moreover, empathetic communication builds rapport, enhancing patient comfort and cooperation. The integration of technical knowledge and interpersonal skills in MHx capture is pivotal in healthcare, directly impacting patient outcomes and healthcare quality.

Large language models (LLMs) have become increasingly popular and utilized in various prompt/response applications such as search engines, customer service, etc. LLMs often include generative AI models trained based on extensive datasets built on historical or bulk general information. To interact with an LLM, a prompt is given to the LLM, and the LLM provides a response back.

### SUMMARY

One aspect of the present embodiments includes the realization that current training schema for medical history (MHx) evaluation interviews are limited, in that each trainee, whether a student or current medical professional, requires a trainer (e.g., a person to assess the performance of the trainee) in the room during the medical interview. However, an additional person in the room with the patient raises additional HIPPA issues that require resolution prior to the interview. Moreover, there is a limited number of trainers available, and therefore not every interview undergoes an evaluation process, because there are not enough trainers/professors available to review every medical interview. Where a trainer reviews a transcript or recording of the trainee's interview with the patient, the trainer is limited to information in the transcript, may exhibit an inherent bias, and may be unaware of cultural norms associated with the interviewee.

The present embodiments solve these problems by implementing an artificially intelligent (AI) medical history evaluation system and associated methods to evaluate multiple social aspects of speech during the medical history evaluation interview, including, but not limited to, politeness, empathy, and the use of jargon or specialized vocabulary. The medical history evaluation interview is automatically transcribed and excerpts from the transcript are analyzed by the AI dialog evaluation system, which provides suggestions on how the trainee may improve their performance in these areas. Many new virtual reality (VR) and avatar-based patient interviewing and physical examination platforms, offer immediate speech-to-text transformation, and provide an output that is suitable for evaluation by an AI-based system.

In certain embodiments, the techniques described herein relate to a method for artificially intelligent medical history interview evaluation, including: receiving captured data of a medical history evaluation interview between a patient and a medical provider; transcribing audio of the captured data into transcribed text; segmenting the transcribed text into segmented lines according to a speaker of the transcribed text within the audio; generating a plurality of prompts, each prompt corresponding to one of a plurality of interview analysis variables, to control a large language model (LLM) to analyze each of the segmented lines in context of the transcribed text; transmitting the plurality of prompts to the LLM; receiving LLM responses from the LLM for each of the plurality of prompts; analyzing the LLM responses with respect to a scoring rubric; and generating a detail report defining performance of the medical provider during interaction between the patient and the medical provider.

In certain embodiments, the techniques described herein relate to a system for artificially intelligent medical history evaluation, including: a capture device configured to capture information an medical history evaluation interview between a patient and a medical provider; an evaluation device having at least one processor and memory storing non-transitory executable instructions that, when executed by the processor operate to control the evaluation device to: receive, from the capture device, captured data of the medical history evaluation interview; transcribe audio of the captured data into transcribed text; segment the transcribed text into segmented lines according to a speaker of the transcribed text within the audio; generate a plurality of prompts, each prompt corresponding to one of a plurality of interview analysis variables, to control a large language model (LLM) to analyze each of the segmented lines in context with the transcribed text; transmit the plurality of prompts to the LLM; receive LLM responses from the LLM for each of the plurality of responses; analyze the LLM responses with respect to a scoring rubric; and generate a detail report defining performance of the medical provider during interaction between the patient and the medical provider.

In certain embodiments, the techniques described herein relate to a method for artificially intelligent debriefing dialog evaluation, including: receiving captured data of a debriefing dialog for a scenario-based medical simulation; transcribing audio of the captured data into transcribed text; segmenting the transcribed text into segmented lines according to a speaker of the transcribed text within the audio; generating a plurality of prompts, each prompt corresponding to one of a plurality of interview analysis variables, to control a large language model (LLM) to analyze each of the segmented lines in context of the transcribed text; transmitting the plurality of prompts to the LLM; receiving LLM responses from the LLM for each of the plurality of prompts; analyzing the LLM responses with respect to a scoring rubric; and generating a detail report defining performance of a trainee demonstrating scenario-based medical simulation based on the debriefing dialog.

### BRIEF DESCRIPTION OF THE FIGURES

FIG. 1 is a schematic diagram showing one example artificial intelligence (AI) dialog evaluation system for evaluating a medical history interview, in embodiments.

FIG. 2 shows example processing of transcribed text of FIG. 1 into segmented text, in embodiments.

FIG. 3 is a schematic diagram illustrating one example detailed report generated by the evaluation device of FIG. 1, in embodiments.

FIG. 4A shows a portion of the detailed report of FIG. 3, illustrating selections of fields to cause pop-out of an additional detail windows, in embodiments.

FIG. 4B shows example content of a first additional detail window of FIG. 4A, in embodiments.

FIG. 4C shows example content of a second additional detail window of FIG. 4A, in embodiments.

FIG. 5 is a flowchart illustrating one example method for artificially intelligent dialog evaluation, in embodiments.

FIGS. 6A and 6B show an example summary report generated by the evaluation device of FIG. 1, in embodiments.

FIG. 7 shows one example supervisor report template used by the evaluation device of FIG. 1 to generate a supervisor summary of the medical history interview, in embodiments.

FIG. 8 shows one example scoring table generated by the scoring module of FIG. 1, in embodiments.

FIG. 9 shows one example summary section generated by the evaluation device of FIG. 1, in embodiments.

FIG. 10 shows one example section of summary report and/or scoring rubric corresponding to a first example category, in embodiments.

FIGS. 11A, 11B, and 11C show one example debriefing dialog between a group of participants in a scenario-based medical simulation as captured by capture device and transcribed into transcribed text.

FIGS. 12A and 12B show a detailed report with a heading block identifying context details of debriefing dialog and example result sections that define performance of a trainee of the scenario-based medical simulation as determined from a debriefing dialog evaluated against scoring rubric, in embodiments.

FIG. 13 shows one example summary report section generated by the LLM and/or the scoring module of the system of FIG. 1 based on analysis of the debriefing dialog, in embodiments.

### DETAILED DESCRIPTION OF THE EMBODIMENTS

It should be noted that the described system and methods do not provide medical analysis of a captured medical history. Rather, the systems and methods disclosed herein analyze a trainee's techniques for capturing the medical history of a patient during an interview with the patient.

A large library of anonymous medical interviews has been accumulated for machine learning. A scoring template, or scoring rubric, and an interactive user interface for individual patient interviews provides each student/trainee with detailed scoring and coaching after the interview is complete. Certain embodiments provide additional real-time feedback, including one or more of audio feedback, visual feedback, and haptic feedback to the interviewee during the interview. The AI dialog evaluation system saves thousands of faculty and instructor hours annually by eliminating the need for manual interview scoring. The AI dialog evaluation system is capable of monitoring multiple trainees simultaneously. The AI dialog evaluation system interacts with large language models (LLMs) in specific ways to decrease the time required to provide feedback, and to reduce/eliminate inter-and intra-evaluator variability and bias.

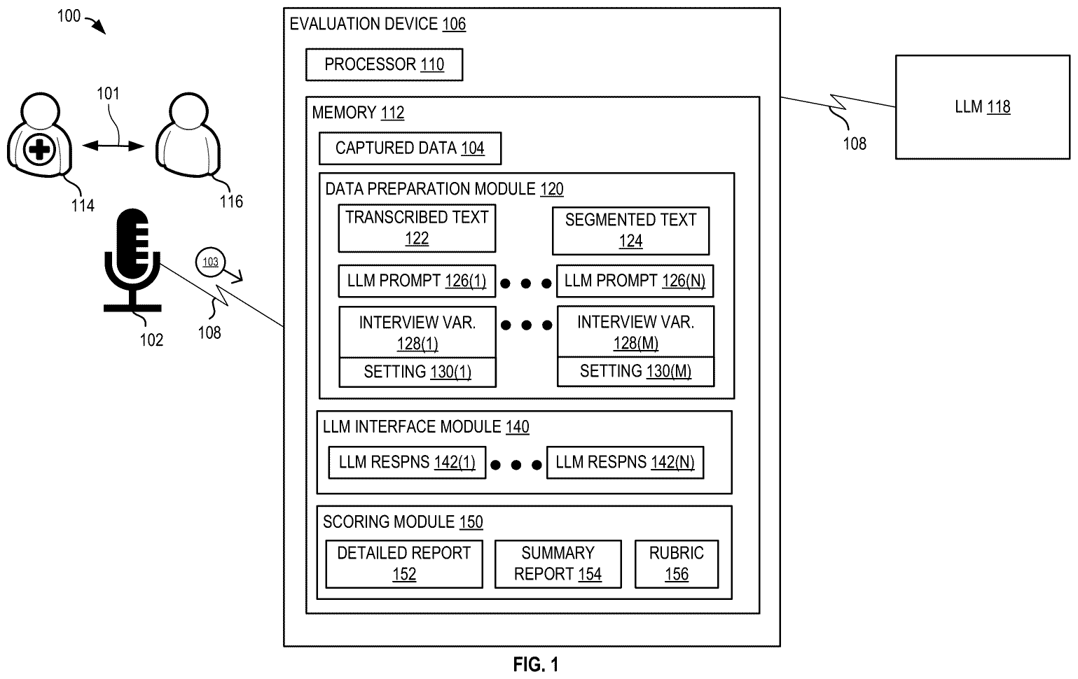

FIG. 1 is a schematic diagram showing one example AI dialog evaluation system 100 for evaluating a medical history interview 101, in embodiments. System 100 includes a capture device 102 that captures audio, video, and/or other audio/visual information, shown as information 103, of medical history evaluation interview 101 between a medical provider 114 and a patient 116. System 100 also includes an evaluation device 106 that includes a processor 110 and a memory 112. Evaluation device 106 may be implemented as one or more of an embedded device, a stand-alone computer, a server, and a cloud service.

Capture device 102 may include any one or both of a microphone and a camera. Capture device 102 may be a stand-alone device, or may be integrated into another device, such as any one or more of a laptop computer, a desktop computer, a smart phone, a tablet, a medical device, and other such devices. In certain embodiments, capture device 102 hosts an application or web-browser that assists in capture of information 103 of interview 101. Capture device 102 sends information 103 to evaluation device 106 where it is stored as captured data 104 (e.g., raw or unmodified digital data corresponding to information 103) in memory 112. In certain embodiments, evaluation device 106 is implemented with capture device 102. Where evaluation device 106 is separate from capture device 102, both capture device 102 and evaluation device 106 may communicate (wirelessly and/or wired) via a network 108. Network 108 is implemented using one or more of Ethernet, Wi-Fi, Cellular, Bluetooth, ANT+, and any other similar wired or wireless connectivity protocol.

Evaluation device 106 includes data, software, and firmware, stored within memory 112, that implements functionality of evaluation device 106 as described herein. Processor 110 may be any type of circuit or integrated circuit capable of performing logic, arithmetic, control, and input/output operations. For example, processor 110 may include one or more of a microprocessor with one or more central processing unit (CPU) cores, a graphics processing unit (GPU), a digital signal processor (DSP), a field-programmable gate array (FPGA), a system-on-chip (SoC), a microcontroller unit (MCU), and an application-specific integrated circuit (ASIC). Processor 110 may also include a memory controller, bus controller, and other components that manage data flow between processor 110, memory 112, and other components connected to processor 110 and evaluation device 106. For example, memory 112 stores non-transitory computer-readable instructions that, when executed by processor 110, control processor 110 to implement the described functionality of evaluation device 106. It should be appreciated that none, all, or some but not all components and/or functionality of evaluation device 106 may be implemented using a cloud computing service, such as Amazon Web Services (AWS), Microsoft Azure, Google Cloud, Hostwinds, Cloudways, Hostinger, and the like.

In one example of operation, capture device 102 is positioned within a patient room, or a training facility, and positioned to monitor interaction between medical provider 114 and patient 116. Patient 116 may be an actual patient, or may be a training patient. In certain embodiments, capture device 102 is a component of a virtual training device, such as a virtual reality (VR) device, where patient 116 is a virtual patient. Examples of VR devices include, but are not limited to, the PCS Spark Virtual Patient System (by Team PCS North America LLC), SimX VR System (by SimX, Inc.), Osso VR Systems (by Osso VR, Inc.), Apple Vision Pro implementing a virtual patient software program, etc.

### Data Preparation for Submission to LLM

Evaluation device 106 communicates (e.g., via network 108) with at least one LLM 118, which is controlled by evaluation device 106 to analyze captured data 104 to provide interview analysis of the monitored interaction between medical provider 114 and patient 116. In certain embodiments, LLM 118 is a locally-installed/managed LLM within evaluation device 106. In other embodiments, LLM 118 is remote from evaluation device 106 and evaluation device 106 communicates with LLM 118 via network 108, whereby evaluation device 106 transmits prompts to, and receives responses from LLM 118 via API calls.

To use LLM 118 efficiently, evaluation device 106 conditions the data used in the interactions with LLM 118. Simply transmitting captured data 104 without preparing captured data 104 would result in inefficient use of LLM 118 and would prevent real-time analysis and feedback of the monitored interaction between medical provider 114 and patient 116. Accordingly, evaluation device 106 includes a data preparation module 120 that performs a transcription of captured data 104 into a transcribed text 122. Data preparation module 120 is software (e.g., computer-readable instructions executable by processor 110) implementing one or more algorithms that control processor 110 to process 104 prior to interaction with LLM 118.

Data preparation module 120 may implement various voice-to-text transcription protocols, known in the art, to transcribe information 103 of captured data 104 into transcribed text 122. Data preparation module 120 then segments transcribed text 122 into a segmented text 124 that associated each line of transcribed text 122 with a given speaker.

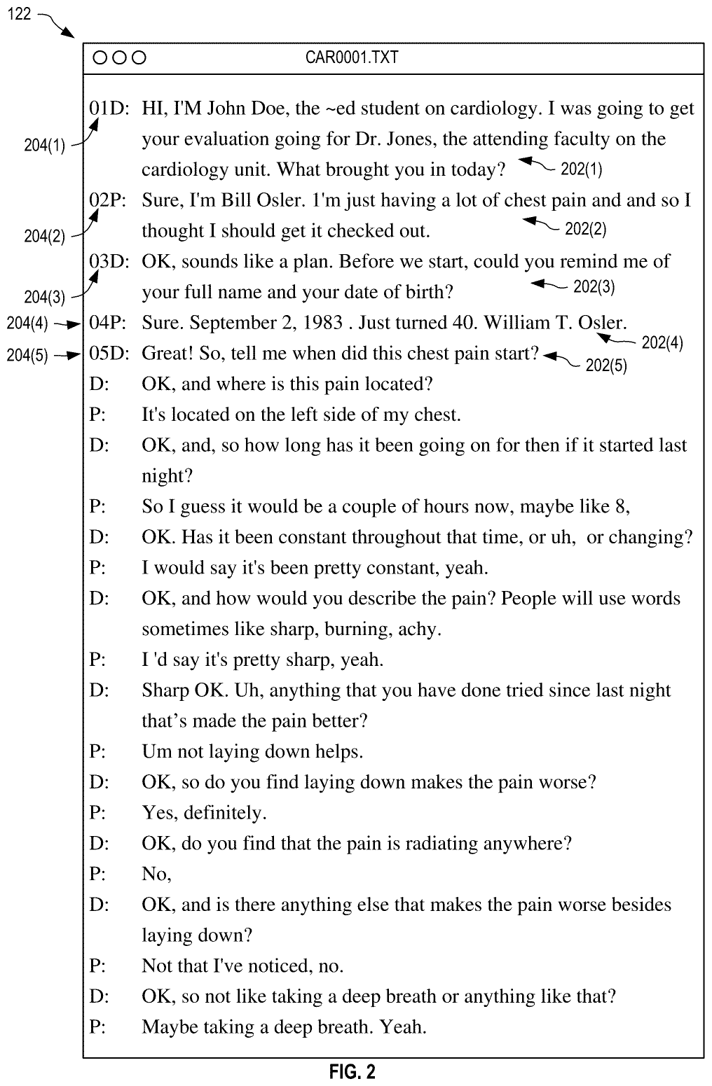

FIG. 2 shows example processing of transcribed text 122 into segmented text 124, in embodiments. In one example of operation, to generate segmented text 124, data preparation module 120 identifies each line, or set of adjacent lines, that is/are associated with a given speaker (e.g., a first speaker is medical provider 114 and a second speaker is patient 116). Each line/set of lines may be assigned a reference number 204 that may be used to identify the line/set of lines subsequently. The example of FIG. 2 shows a first five segmented lines 202(1)-202(5), that are lines of transcribed text 122 processed into segmented text 124, whereas the remaining lines of transcribed text 122 are not fully segmented. The reference numerals 204(1)-(5) of segmented lines 202(1)-(5) each include one of a “P” to identify patient 116 as speaker, and “D” to identify medical provider 114 (e.g., doctor) as a speaker. Although shown as a complete set of transcribed lines, processing of captured data 104 into transcribed text 122 and segmented text 124 may occur in real-time as each speaker is talking. That is, processing of captured data 104 does not wait until medical history interview 101 is completed.

Evaluation device 106 is configured with a set of interview analysis variables 128, each with a corresponding settings module 130, that define metrics for analyzing medical history interview 101, and in particular for analyzing segmented text 124. For segmented lines 202, data preparation module 120 generates one or more prompts 126(1)-126(N) for use with LLM 118, where each prompt 126 represents a query that causes LLM 118 to analyze medical history interview 101 according to one or more interview analysis variables 128. Prompts 126 are generated to cause LLM 118 to analyze each segmented line 202 of segmented text 124 in the context of interview 101. For example, a first prompt 126(1) may query segmented line 202(1) “01D” against one or more interview analysis variables 128. A second prompt 126(2) may query line “02P” against one or more of interview analysis variables 128. Data preparation module 120 generates prompts 126 such that each of segmented lines 202 of segmented text 124 are associated with at least one prompt 126.

### Interview Analysis Variables

Interview analysis variables 128 may be configured by an administrator or may be generated at least in part by an automated process. In one example, interview analysis variables 128 are generated by controlling an LLM to identify appropriate metrics for analyzing a medical history interview by analyzing one or more of textbooks, well-established medical sources, and so on. In one example, Bate's Guide to Physical Examination and Medical History, Robert Wood Johnson Foundation (2017): Patient Centered Medicine: Guidebook for History Taking and Physical Examination, the American College of Physicians syllabi, WHO, and LCME were analyzed to determine interview analysis variables 128(1)-128(M). For example, each interview analysis variable 128 may defining analysis parameters for LLM 118 based on one or more of: (1) Healthcare Provider identification (ID); (2) Patient/Client Subject Identification; (3) Comfort Check; (4) Proper Mode of Addressing Patient; (5) Chief Complaint; (6) Health Provider Identification (HPI); (7) Past Medical History (PMHx); (8) Family and Social History; (9) Familial, Genetic History and Travel History; (10) Review of Systems (ROS); (11) Rephrasing; (12) Reformulation; (13) Empathetic expression; (14) Politeness score; (15) Medical or technical jargon; and/or (16) Wrapping up & Conclusion.

Each variable of interview analysis variables 128 may be further configurable through an associated settings module 130. Settings module 130 is, for example, a python code module, or the like, that includes a definition of constraints for the associated interview analysis variable 128. As an example, where interview analysis variable 128 corresponds to “medical or technical jargon,” the associated settings module 130 may include a list of medical or technical terms, or a list of approved resources to use when analyzing a given term or set of terms of segmented text 124. Each interview analysis variable 128 may include the corresponding settings module 130. Alternatively, settings module 130 may be transmitted to LLM 118 and used by LLM 118 for configure to set a context for analyzing segmented text 124, or certain segmented lines 202, for each prompt 126 received by the LLM 118.

Advantageously, since each interview analysis variables 128 has a corresponding settings module 130, each setting module 130 is easier to manipulate and change, making changing of a given setting efficient. For example, since cultural norms may vary by country, state, region, etc., settings module 130 corresponding to interview analysis variables 128 for empathy, or proper mode of addressing the patient, or any other variable affected by cultural norms, may be individually configured by adjusting associated settings module 130 accordingly. Thus, system 100 is easily adapted for use in different regions, and/or for different characteristic (e.g., gender, sexual preference, race, ethnic identity, etc.) of one patient versus another patient.

### LLM Interface Module

Evaluation device 106 may also include an LLM interface module 140. LLM interface module 140 may be implemented as software (e.g., non-transitory computer readable instructions executable by processor 110) that controls processor 110 to implement the following functionality.

LLM interface module 140 queues each prompt 126, received from data preparation module 120, for example, for transmittal to LLM 118. In certain embodiments, each prompt 126 is submitted sequentially to LLM 118. In other embodiments, each prompt 126 is transmitted to an individual instance of LLM 118, such that prompts 126 are transmitted in parallel. The number of individual instances of LLM 118 may be configured based on the types of prompt 126, the types of interview analysis variables 128, etc. Advantageously, the use of multiple instances of LLM 118 and parallel transmission of prompts 126 enables faster interfacing with the LLM 118 such that results and analysis of the medical interview are provided in an expedited manner. In certain embodiments, evaluation device 106 uses sixteen instances of LLM 118 such that the sixteen different interview analysis variables 128, discussed above, are processed simultaneously, such that segmented text 124 of medical provider 114 is sampled as many as 36,000 times for transcribed text 122 of one-hundred lines. Sequential query/response processing by LLM 118 may take twenty minutes or more; however, by grouping prompts 126 and using multiple instances of LLM 118 to query the prompts in parallel, the query/response processing time may be reduced to less than one minute.

LLM interface module 140 receives LLM responses 142(1)-142(N) generated by LLM 118 for each prompt 126(1)-126(N), and stores each LLM response 142 in memory 112. Each LLM response 142 includes an identification of interview analysis variable 128 for which it was generated. For example, LLM response 142(1) may identify any of interview analysis variables 128 based on the one or more interview analysis variable defined by prompt 126(1). Where prompt 126 identifies more than one interview analysis variable 128, LLM 118 generates more than one LLM response. Thus, there may be more LLM responses 142 than prompts 126, since each LLM response 142 is associated with one interview analysis variable 128.

Certain LLM responses 142 may be “binary” (e.g., “yes” or “no”) for a given interview analysis variable 128. For example, where interview analysis variable 128 relates to whether or not patient 116 is properly identified, the corresponding LLM response 142 includes “yes” when LLM 118 determines that patient 116 was properly identified and “no” when patient 116 was not properly identified. Certain LLM responses 142 that are binary may include additional information such as indicating a portion of transcribed text 122 that satisfied (or did not satisfy) the corresponding interview analysis variables 128. Continuing the above example, LLM response 142 may include reference number 204(2) to indicate that segmented line 202(2) included a proper identification of patient 116.

Certain LLM responses 142 may be a scaled responses. Scaled responses may provide a sliding score for a given interview analysis variable 128. As an example, the “Empathy” and/or “Politeness” variable may include a sliding scale indicative of a perceived level of empathy and/or politeness. To generate a scaled response, LLM 118, or LLM interface module 140, may generate a series of prompts 126 for the corresponding interview analysis variables 128, as follows. A first scaled-response prompt 126 is generated to include at least one segmented line 202 for analysis. A second scaled-response prompt 126 is associated with the first scaled-response prompt to query LLM 118 to provide a first example text scoring at a low end of the scale. A third scaled-response prompt 126 associated with the first and second scaled-response prompts queries LLM 118 to provide a second example text scoring at a high end of the scale. A fourth scaled-response prompt 126 associated with the first, second, and third scaled-response prompts queries LLM 118 to provide a rating of the segmented line, the first example text, and the second example text on the scale. In certain embodiments, the rating is generated by LLM 118 using a cosine similarity function. The second and third scaled responses may be configured according to cultural norms associated with patient 116 as well, by utilizing associated settings module 130 to alter the requirements of LLM 118 to meet those cultural norms.

### Scoring Module

Evaluation device 106 may also include a scoring module 150. Scoring module 150 may be implemented as software (e.g., non-transitory computer readable instructions executable by processor 110) that controls processor 110 to implement the following functionality. Scoring module 150 receives LLM responses 142 stored in memory 112 by LLM interface module 140 and configures them into a detailed report 152 based on a scoring rubric 156. Scoring rubric 156 corresponds to interview analysis variables 128, for example, and may provide a template for generating detailed report 152. In certain embodiments, detailed report 152 is an interactive scoring rubric 156 that includes information from LLM responses 142.

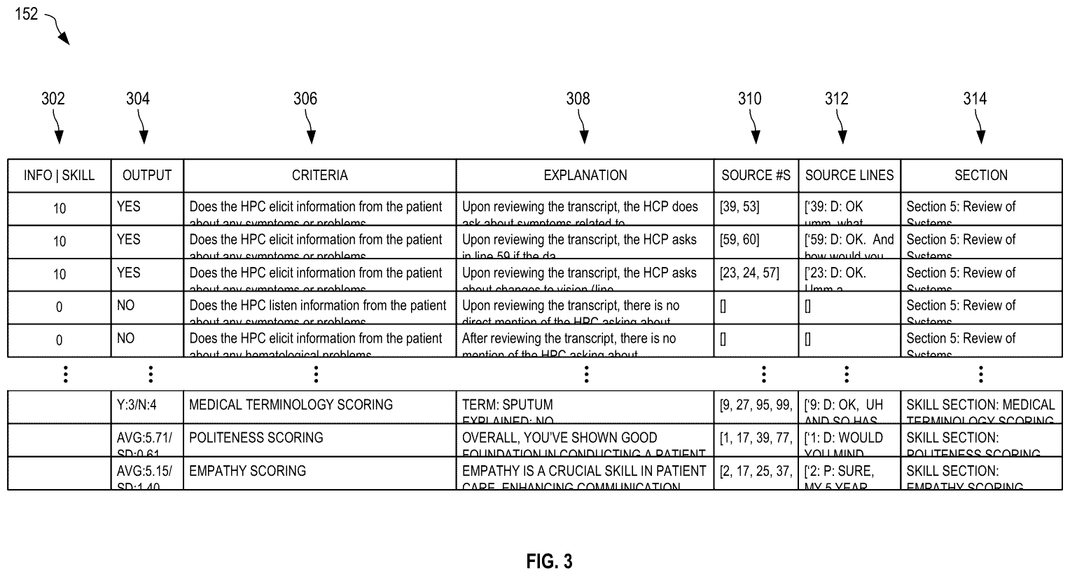

FIG. 3 is a schematic diagram illustrating one example detailed report 152 generated by evaluation device 106 of FIG. 1, in embodiments. Detailed report 152 may be an interactive rubric where certain fields may be selected, via interaction with detailed report 152 on a computing device, to view further detail on how and/or why LLM response 142 associated with that field was generated. Detailed report 152 is formed with a plurality of columns including an info/skill column 302, an output column 304, a criteria column 306, an explanation column 308, a source number column 310, a source lines column 312, and a section column 314. Each row of detailed report 152 corresponds to one defined criteria of criteria column 306.

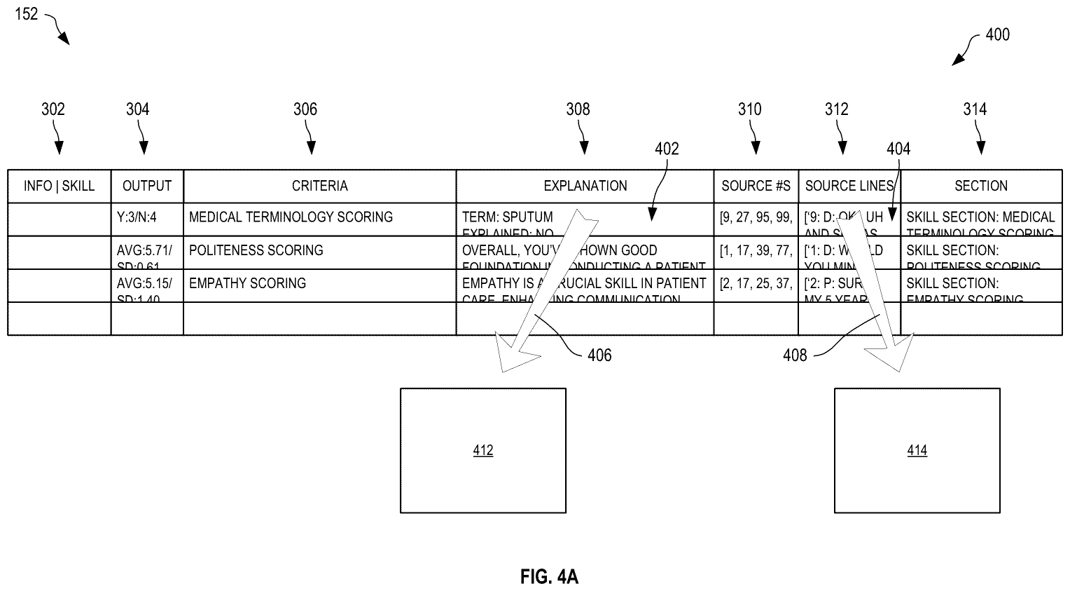

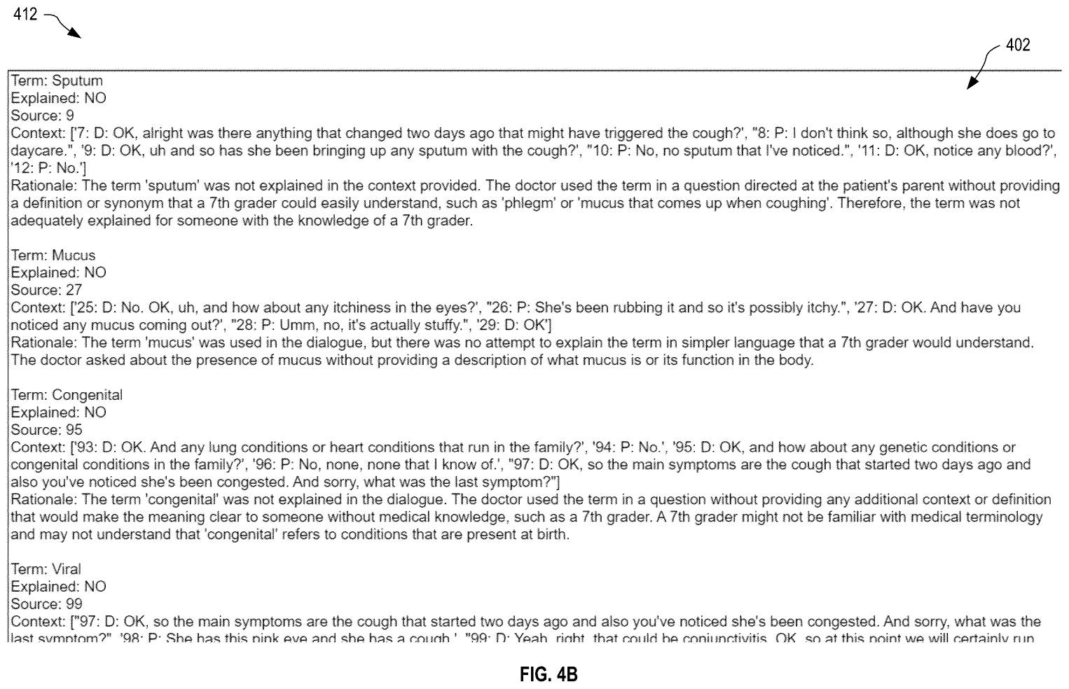

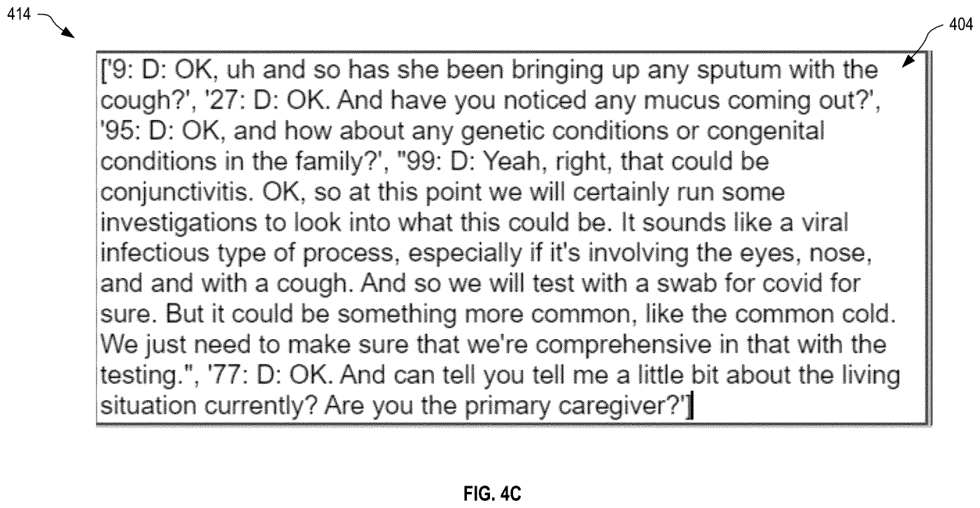

FIGS. 4A, 4B, and 4C are schematic diagrams illustrating expansion of two fields 402 and 404 of detailed report 152 in response to selection by a user, to show additional detail, in embodiments. FIG. 4A shows a portion 400 of detailed report 152 where the user has selected field 402, causing pop-out 406 of an additional detail window 412 and selection of field 404, causing pop-out 408 of an additional detail window 414. FIG. 4B shows example content of additional detail window 412 of FIG. 4A, illustrating details of terms that were not visible on portion 400 of LLM response 142, in embodiments. FIG. 4C shows example content of additional detail window 414, illustrating many lines of segmented text 124 that are relevant to the corresponding line of portion 400 of detailed report 152, in embodiments. Advantageously, by providing interactive detailed report 152, a user (e.g., medical provider 114) may see details of selected fields that would otherwise not be displayed.

In this example, field 402 corresponds to a detailed explanation of a medical terminology scoring criteria and field 404 corresponds to a corresponding portion of segmented text 124. Additional detail window 412 displays the detailed explanation corresponding to field 402 and, in this example, provides rationale of why the “Sputum” term use in the segmented text 124 indicated a poor score, notably that “the doctor used the term in a question directed at the patient's parent without providing a definition or synonym that a 7^{th} grader could easily understand, such as ‘phelgm’ or ‘mucus that comes up when coughing’. Therefore the term was not adequately explained for someone with the knowledge of a 7^{th} grader.” Particularly, LLM 118 has identified the term “Sputum” as medical jargon, and as a likely term that is unknown to the patient. For example, the associated settings module 130 corresponding to interview analysis variables 128 for the medical terminology scoring defines that LLM 118 compare terminology to terms known to a person with the knowledge of a 7^{th} grader. Associated settings module 130 may be configured with parameters other than the knowledge of a 7^{th} without departing from scope hereof. The user selects field 404 to view a portion of segmented text 124 associated with the given term/source for that criteria. Fields 402 and 404 are used for examples, and other fields of 152 may be similarly selected to show corresponding additional detail windows.

Output column 304, which may also be referred to as a score column, may display either binary or scaled values as discussed above for each variable (e.g., interview analysis variables 128). The criteria includes a definition of required criteria associated with each interview analysis variables 128. Criteria column 306 may display information identified by LLM 118 analyzing the context of transcribed text 122 (e.g., the entire transcribed text as currently determined), particularly where each prompt 126 includes the specific line within segmented text 124 to be analyzed, as well as the remainder of the transcribed text 122. Explanation column 308 may display a description provided by LLM 118 within LLM response 142 of how and why the given criteria/interview analysis variables 128 was analyzed and why the displayed score in output column 304 was provided. Source number column 310 and source lines column 312 definition the location of the processed text within segmented text 124 and/or transcribed text 122 that led to the results displayed in the fields of criteria column 306 and explanation column 308.

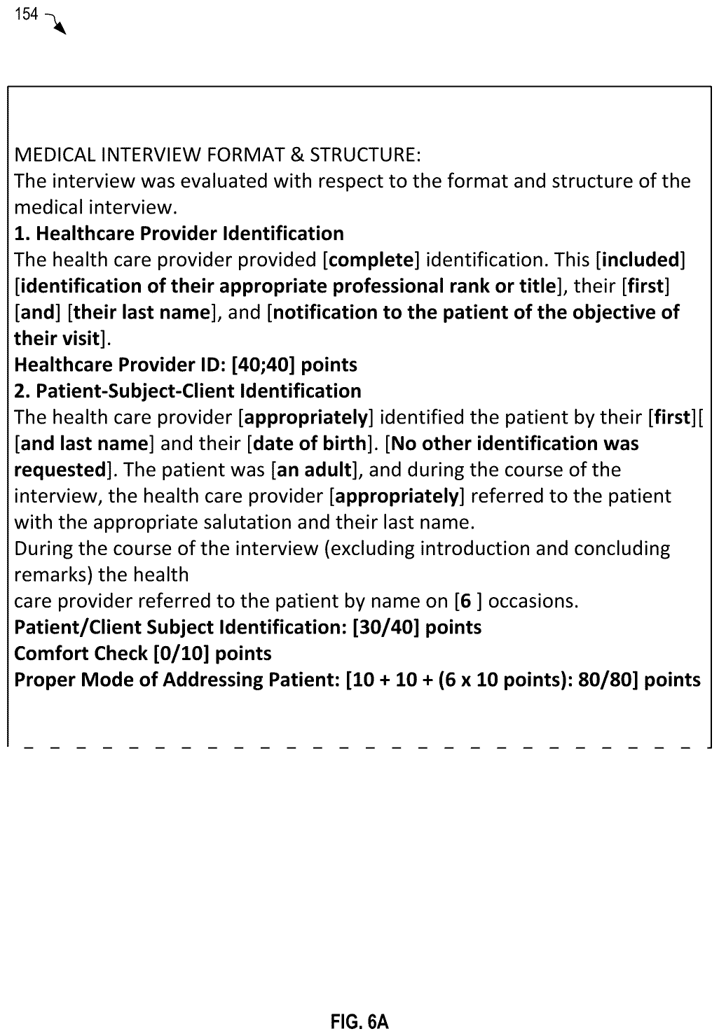

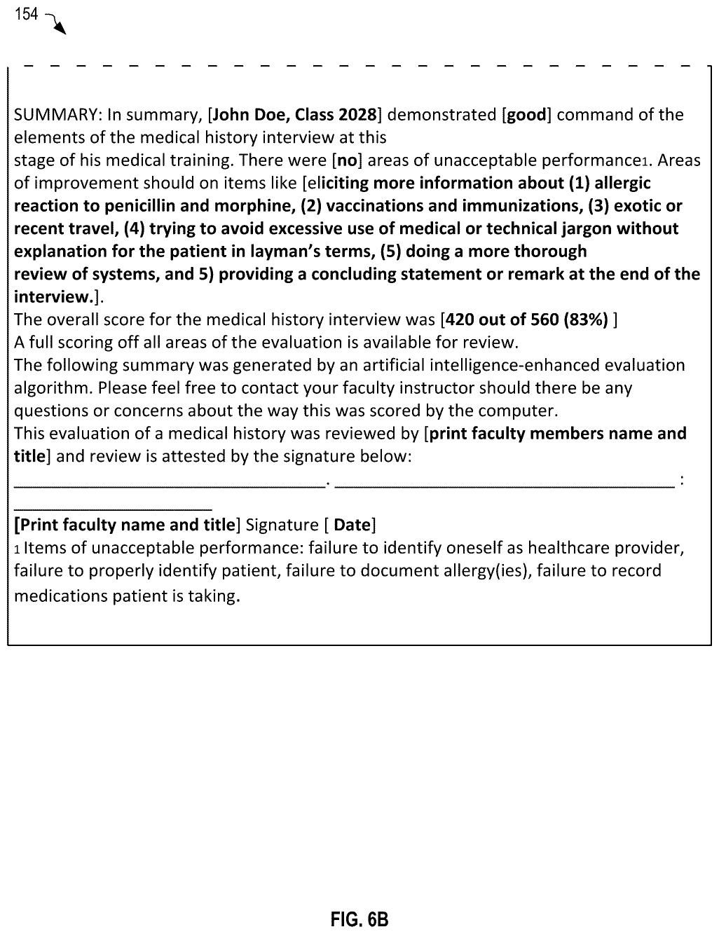

Scoring module 150 may further generate a summary report 154 (see FIG. 1). Appendix B, attached hereto, provides an example summary report 154. It includes a scoring section including numerical scores for one or more of the interview analysis variables 128, as well as a summary section. The summary section may be a plain-language text generated via a query/response process with LLM 118. The summary section may further be generated using a standard format where LLM 118 and/or scoring module 150 are used to complete specific fields within the standard format. FIGS. 6A and 6B show an example summary report 154 generated by evaluation device 106 of FIG. 1 with a summary section where the language in brackets [] is inserted using LLM 118 and/or scoring module 150.

As discussed above, evaluation device 106 may monitor the interaction between medical provider 114 and patient 116 in real time. The use of LLM 118 may additionally occur in real time as the interview is occurring. If the generated detailed report 152 and or summary report 154 indicates a poor score, the evaluation device 106 may interact with a device worn by medical provider 114, such as a watch or smartphone, and provide real-time feedback to the medical provider 114 during the interaction between medical provider 114 and patient 116. Additionally or alternatively, where the score generated by scoring module 150 is below a predefined threshold, the summary report 154 may include a further detailed section that includes description of how the medical provider 114 could have altered the interaction between medical provider 114 and patient 116.

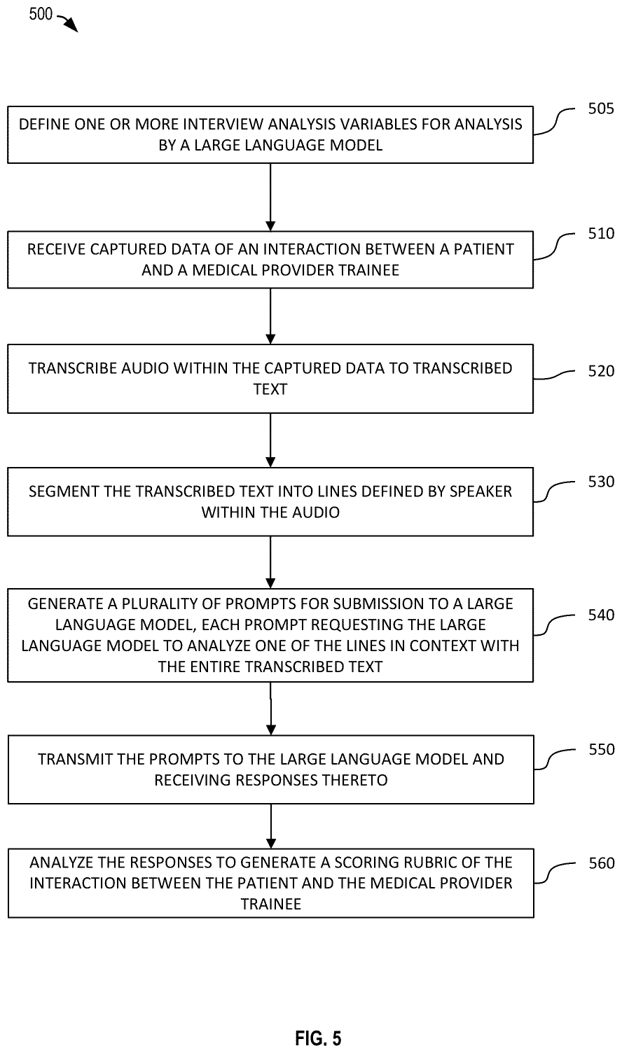

FIG. 5 is a flowchart illustrating one example method 500 for artificially intelligent dialog evaluation of a medical history interview. Method 500 may be implemented using one or more components of system 100, including but not limited to evaluation device 106 and LLM 118. Method 500 includes one or more of the following blocks.

Block 505 is optional in that block 505 may be performed external to method 500. In block 505, method 500 defines one or more interview analysis variables to be analyzed by the LLM. In one example of block 505, for one or more of interview analysis variables 128, one or more associated settings modules 130 are defined, as discussed above, and transmitted by evaluation device 106 to LLM 118 to configure the LLM for analysis of each prompt 126.

In block 510, method 500 receives captured data of an interaction between a patient and a medical provider trainee. In one example of block 510, evaluation device 106 receives captured data 104 from capture device 102 and stores captured data 104 within memory 112.

In block 520, method 500 transcribes audio within the captured data to transcribed text. In one example of block 520, data preparation module 120 generates transcribed text 122, which is stored within memory 112.

In block 530, method 500 segments the transcribed text into lines defined by speaker within the audio. In one example of block 530, data preparation module 120 segments transcribed text 122 in to segmented text 124, which is stored within memory 112.

In block 540, method 500 generates a plurality of prompts for submission to an LLM, each prompt requesting the LLM to analyze one of the lines in context with the entire transcribed text. In one example of block 540, data preparation module 120 generates one or more prompts 126, each based on one or more of interview analysis variables 128.

In at least one embodiment, generating a plurality of prompts 126 includes generating a prompt for a binary analysis of one or more interview analysis variables 128.

In at least one embodiment, generating a plurality of prompts 126 includes generating a prompt for a scaled analysis of one or more interview analysis variables 128. In at least one embodiment, generating a prompt 126 for a scaled analysis includes: generating a first scaled-response prompt 126 to include a corresponding segmented text 124 to be analyzed; generating a second scaled-response prompt 126, associated with the first scaled-response prompt, querying LLM 118 to provide an example of a low-scoring end of the scale; generating a third scaled-response prompt 126, associated with the first and second scaled-response prompts, querying LLM 118 to provide an example of a high-scoring end of the scale; and generating a fourth scaled-response prompt 126 associated with the first, second, and third scaled-response prompts, querying LLM 118 to provide a rating of the provided segmented text 124 on a given scale. The rating may include a cosine similarity analysis.

In block 550, method 500 transmits the prompts to the LLM and receiving responses thereto. In one example of block 550, evaluation device 106 transmits prompt 126 to LLM 118, receives LLM responses 142 from LLM 118, and stores each LLM response 142 within memory 112. Transmitting the prompts 126 to LLM 118 may include transmitting one or more of prompts 126 in parallel to individual instances of LLM 118, thereby analyzing the prompts concurrently to receive LLM responses 142 quicker, as compared to sending prompts 126 sequentially to LLM 118.

In block 560, method 500 analyzes the responses to generate a scoring rubric of the interaction between the patient and the medical provider trainee. In one example of block 560, scoring module 150 generates one or more of detailed report 152 and summary report 154 as discussed above. Evaluation device 106 may output the detailed report 152 and/or summary report 154 to an external device for evaluation by medical provider 114.

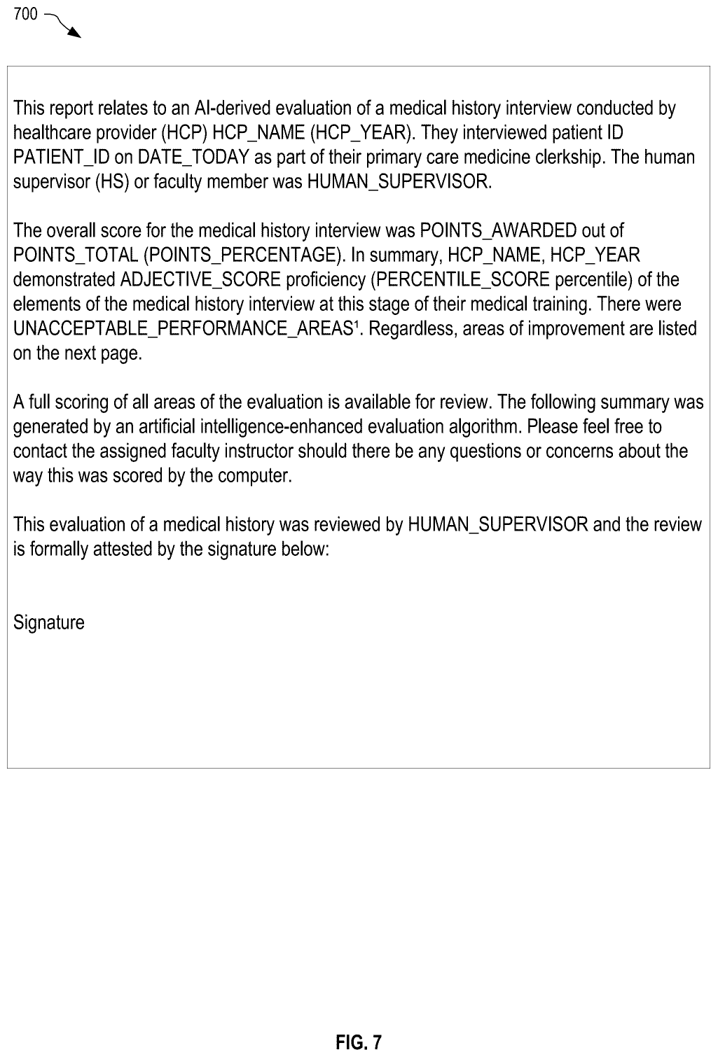

FIG. 7 shows one example supervisor report template 700 that is used by evaluation device 106 of FIG. 1 to generate a supervisor summary of the medical history interview 101, in embodiments. Capital letters represent fields that are automatically completed by scoring module 150 based on LLM response 142 and/or detailed report 152. As appreciated, system 100 may be configured to generate many types of report based on information in detailed report 152.

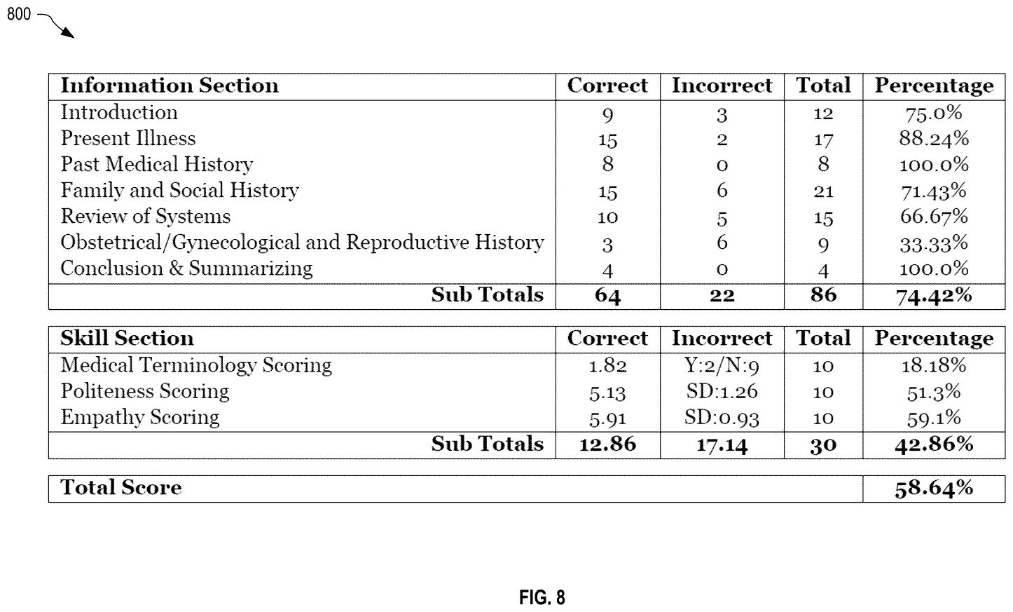

FIG. 8 shows one example scoring table 800 generated by scoring module 150 of FIG. 1, in embodiments. Scoring table 800 provides an overview of the evaluated performance of medical provider 114 during medical history interview 101. Scoring table 800 may form part of summary report 154.

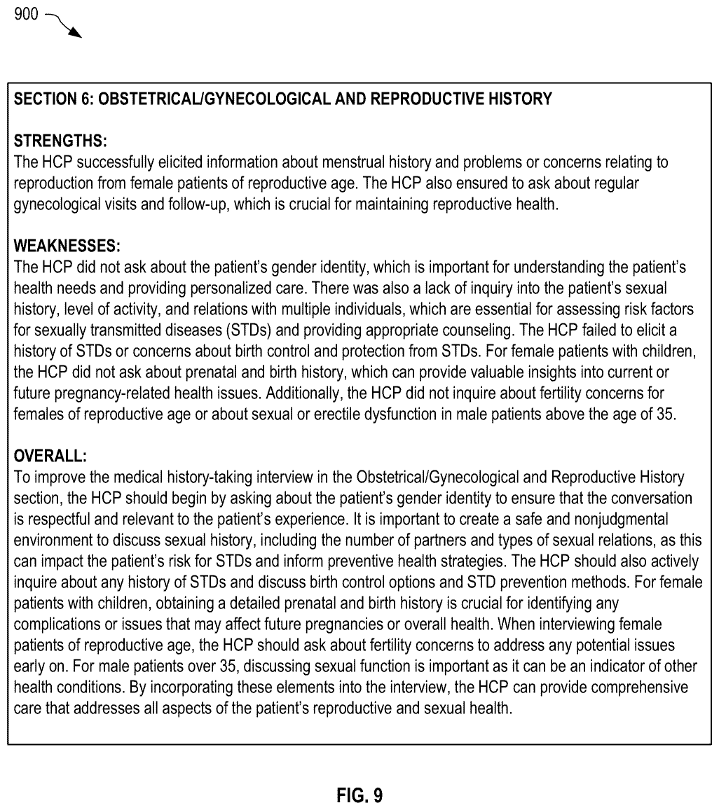

FIG. 9 shows one example summary section 900 generated by evaluation device 106 of FIG. 1, in embodiments. In this example, summary section 900 relates to a section titled “Obstetrical/Gynecological And Reproductive History.” Evaluation device 106 may generate summaries of other sections of scoring rubric 156. These summaries provide feedback on strengths, weaknesses, and overall performance of 114 during medical history interview 101.

System 100 is versatile and adaptable to evaluate different human interactions and provide feedback and reports on performance of a trainee. The following example illustrates this versatility by adapting system 100 for evaluating debriefing techniques for scenario-based medical simulations (SBMS).

### Artificially Intelligent Comprehensive Debriefing Assessment Tool

Traditionally, multiple inventories and batteries have been used to assess how educators and facilitators conduct debriefing sessions with healthcare students after simulation scenarios. These tools, designed to “train the trainer,” typically involve another senior educator or facilitator observing the SBMS debriefing in real-time or through a delayed video recording, using inventory or paper-based scoring sheets. However, this process is both time-consuming and subject to evaluator bias.

The process of debriefing involves reviewing a conversation between two or more people as they evaluate and review a simulated event or activity in which participants explore, analyze, and synthesize their actions, their thought processes, their emotional states, and other information to improve performance in real-life situations. High participant engagement is a hallmark of strong debriefings because it leads to deeper levels of learning and increases the likelihood of transfer to the clinical setting.

Existing tools evaluate the strategies and techniques used to conduct debriefings by examining concrete behaviors, based on the theory that people learn and apply information when they have an experiential context in which they can use it. However, in almost all situations, debriefing assessment is carried out for the benefit of the person leading the debriefing, usually a senior facilitator or faculty member.

To address the limitations of the prior art, system 100 is adapted to evaluate the transcript of the debriefing session by combining best practices of validated debriefing tools and thereby create a superior amalgam of attributes. Accordingly, system 100 is configured with specific criteria and algorithms to provide a comprehensive assessment of debriefing conversations. System 100 evaluates debriefing transcripts and assigns scores across elements considered critical to debriefing technique and outcome. These elements align with the key objectives of SBMS debriefing, including education and skill development, patient safety and quality improvement, professional development, research and development, and health system integration. The use of AI-powered analysis by system 100 offers several advantages over traditional methods. System 100 provides a highly individualized evaluation with coaching suggestions, custom-tailored to the particular debriefing and facilitator. Moreover, system 100 delivers feedback directly to the trainee (e.g., the individual being evaluated), eliminating the need for assessment by another person. Advantageously, this approach not only saves time but also makes the critique and evaluation free from individual bias and ensures privacy in conveying the results to the trainee. By leveraging AI technology, system 100 addresses the limitations of conventional assessment methods while providing a more efficient, consistent, and objective evaluation of debriefing sessions. Accordingly, system 100 significantly enhances the quality of debriefing in medical simulation education, ultimately contributing to improved learning outcomes for trainees and better patient care. System 100 is adapted to apply specific categories and schema for evaluation of debriefing performance without bias. However, it's important to note that while system 100 represents a promising advancement, human expertise and judgment still play a crucial role in medical education.

In one example, interview analysis variables 128 and associated settings module 130 are adapted to allow data preparation module 120 and LLM interface module 140 to control LLM 118 to analyze a debriefing dialog based on the following categories: (1) Identification section for the session; (2) Structure of lesson (10% of composite scoring); (3) Style of delivery (15% of composite score); (4) Professional demeanor (10% of composite score); (5) Use of instructional tools (15% of composite score); (6) Student engagement (20% of composite score); (7) The attentiveness of students to active learning (20% of composite score); and (8) Cumulative Impressions (10% of composite score). As appreciated, interview analysis variables 128 and associated settings module 130 may be configured with more or fewer categories without departing from the scope hereof. System 100 is adapted to simplify scoring into range of 1-5, where 1 represents poor, 2 represent fair, 3 represents good, 4 represents very good and 5 represents excellent. For each of the above categories, LLM 118 (e.g., implemented using ChatGPT4) is trained using specific criteria and examples of how to assign a score to a training debriefing sessions.

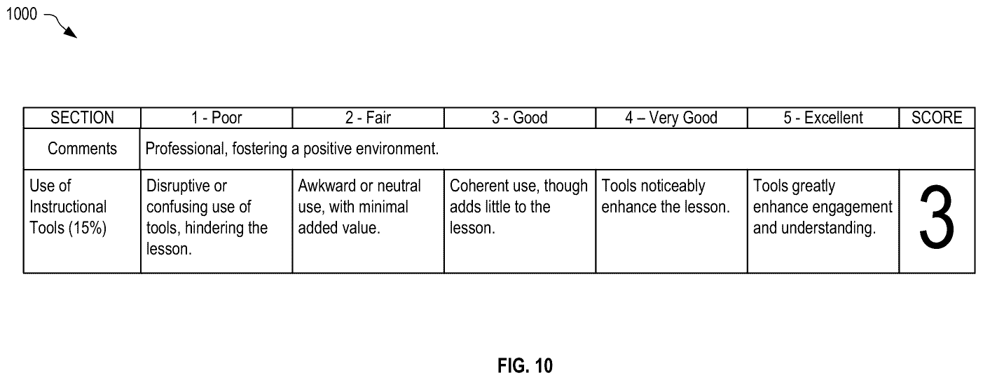

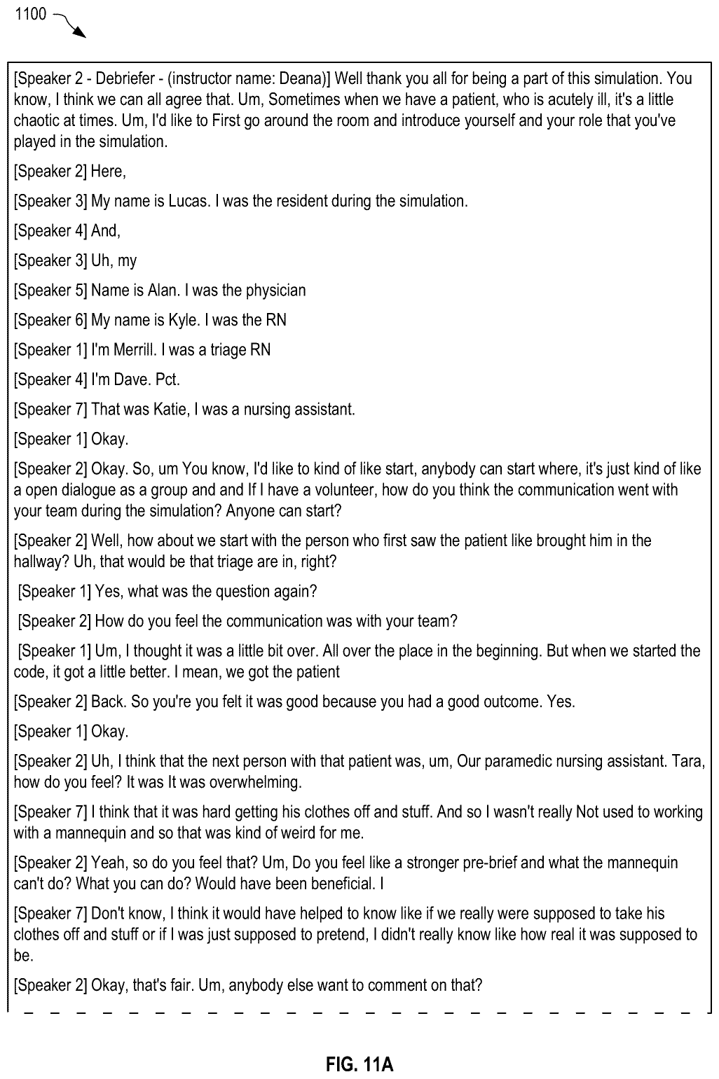

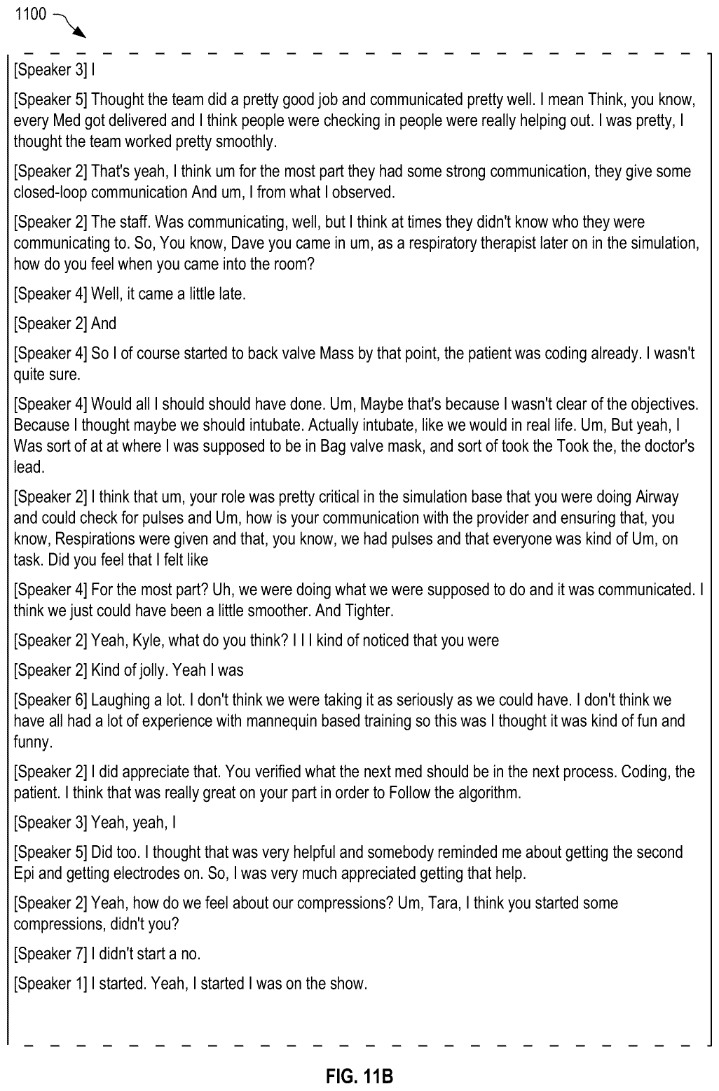

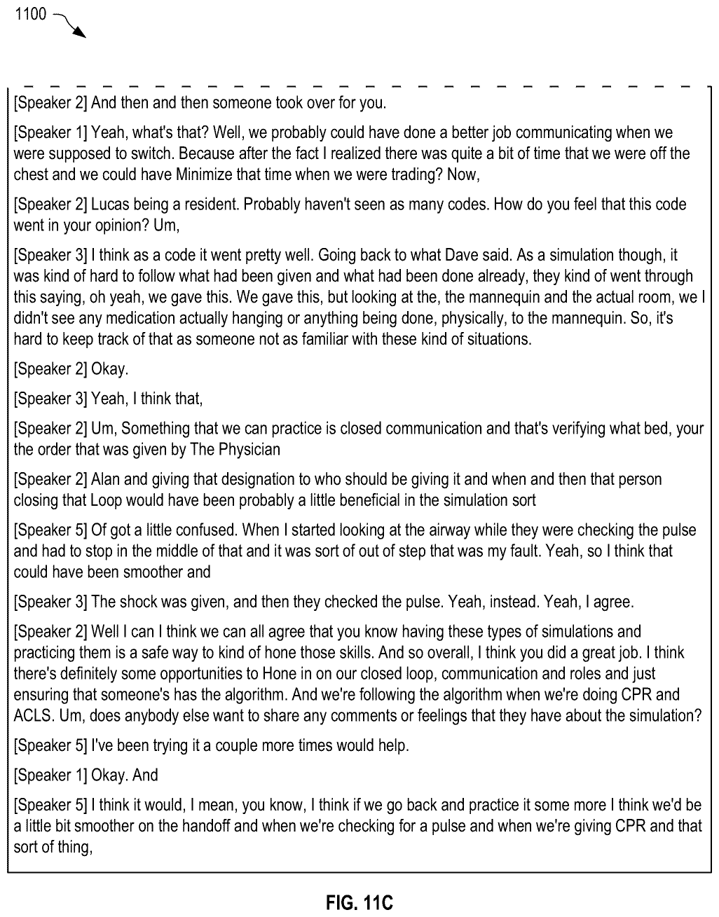

FIG. 10 shows one example section 1000 of summary report 154 and/or scoring rubric 156 corresponding to a first example category. FIGS. 11A, 11B, and 11C show one example debriefing dialog 1100 between a group of participants in a SBMS as captured by capture device 102 and transcribed into transcribed text 122. In this embodiment of system 100, LLM 118 is trained to analyze debriefing dialog 1100 against scoring rubric 156 which is defined based on the categories listed above. Since system 100 is adaptable to use composite sections, it may also be adapted to incorporate any new useful evaluations that appear in the public domain. Advantageously, system 100 may evolve as assessment methodologies are developed and disclosed in the literature. One significant advantage of system 100, because of its AI driven assessment, is that it is free of inter-rater variability and free of individual bias.

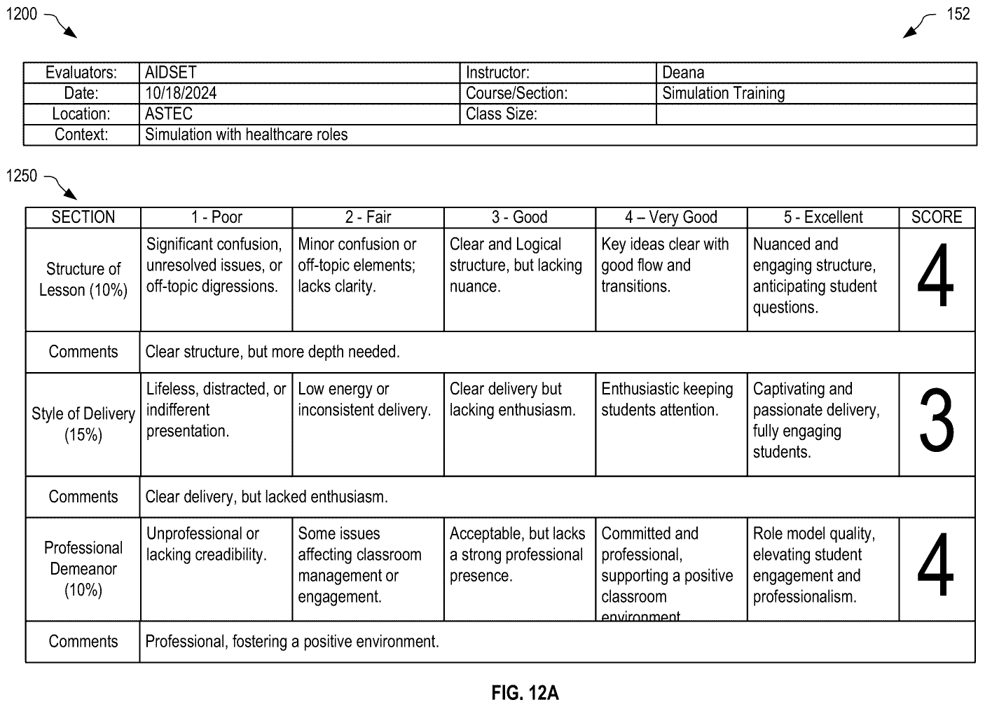

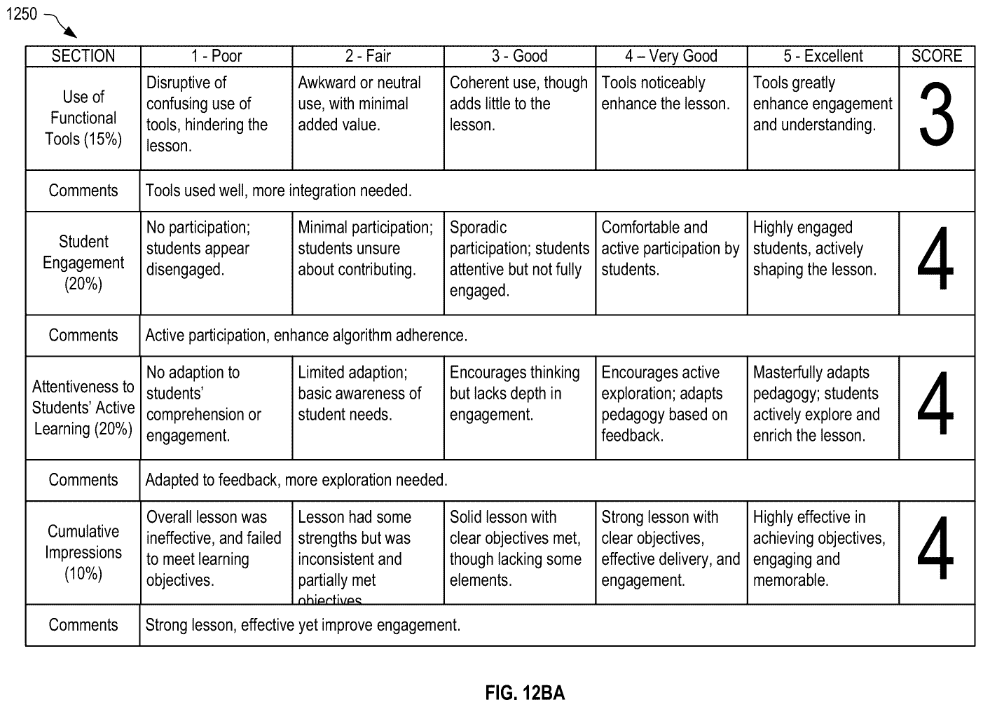

FIGS. 12A and 12B show one example detailed report 152 generated by scoring module 150 using scoring rubric 156 and based on LLM responses 142 resulting from evaluation of transcribed text 122 by LLM 118 in response to generated prompts 126, interview analysis variables 128, and corresponding settings modules 130. System 100 is adapted to implement a composite of four debriefing assessment or evaluation tools and may be referred to as a “Composite Debriefing Assessment Tool” (CDAT). FIG. 12A shows detailed report 152 with a heading block 1200 identifying context details of debriefing dialog 100 and examples result sections 1250 that define performance results determined from debriefing dialog 1100 against scoring rubric 156, for example.

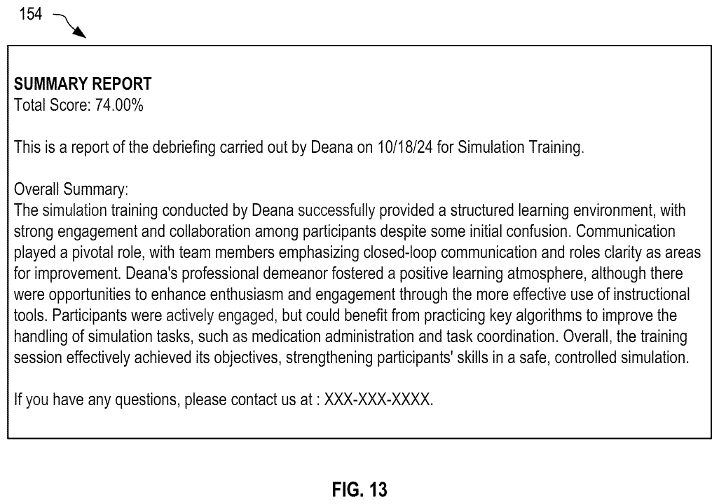

FIG. 13 shows one example summary report 154 section generated by LLM 118 and/or scoring module 150 based on analysis of debriefing dialog 1100 based on prompt 126, interview analysis variables 128, associated settings module 130, and scoring rubric 156.

Advantageously, system 100 generates detailed report 152 and summary report 154 without bias. Accordingly, system 100 provides an improvement over the art by removing bias that is unavoidably subconsciously imparted by a human evaluator.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for artificially intelligent medical history interview evaluation, comprising:
receiving captured data of a medical history evaluation interview between a patient and a medical provider;
transcribing audio of the captured data into transcribed text;
segmenting the transcribed text into segmented lines according to a speaker of the transcribed text within the audio;
generating a plurality of prompts, each prompt corresponding to one of a plurality of interview analysis variables, to control a large language model (LLM) to analyze each of the segmented lines in context of the transcribed text;
transmitting the plurality of prompts to the LLM;
receiving LLM responses from the LLM for each of the plurality of prompts;
analyzing the LLM responses with respect to a scoring rubric; and
generating a detail report defining performance of the medical provider during interaction between the patient and the medical provider.

2. The method of claim 1, the plurality of interview analysis variables each defining analysis parameters for the LLM.

3. The method of claim 1, further comprising transmitting one or more settings, respectively defined for each of the plurality of interview analysis variables, to the LLM to configure the LLM to analyze the segmented line in context with the transcribed text.

4. The method of claim 1, wherein transmitting the prompts to the LLM includes transmitting at least two of the prompts in parallel to at least two different instances of the LLM.

5. The method of claim 1, the generating a plurality of prompts comprises generating a prompt for a binary analysis of one or more of the plurality of interview analysis variables.

6. The method of claim 1, the generating a plurality of prompts comprises generating a prompt for a scaled analysis of one or more of the plurality of interview analysis variables.

7. The method of claim 6, the generating a prompt for a scaled analysis comprises:
generating a first scaled-response prompt including the segmented line;
generating a second scaled-response prompt associated with the first scaled-response prompt querying the LLM to provide an example of a low-scoring end of a scale;
generating a third scaled-response prompt associated with the first and second scaled-response prompts querying the LLM to provide an example of a high-scoring end of the scale; and
generating a fourth scaled-response prompt associated with the first, second, and third scaled-response prompts querying the LLM to provide a rating on a given scale.

8. The method of claim 7, wherein the rating is a cosine similarity analysis between LLM responses associated with the first, second, and third scaled-response prompts.

9. A system for artificially intelligent medical history evaluation, comprising:
a capture device configured to capture information an medical history evaluation interview between a patient and a medical provider;
an evaluation device having at least one processor and memory storing non-transitory executable instructions that, when executed by the processor operate to control the evaluation device to:
receive, from the capture device, captured data of the medical history evaluation interview;
transcribe audio of the captured data into transcribed text;
segment the transcribed text into segmented lines according to a speaker of the transcribed text within the audio;
generate a plurality of prompts, each prompt corresponding to one of a plurality of interview analysis variables, to control a large language model (LLM) to analyze each of the segmented lines in context with the transcribed text;
transmit the plurality of prompts to the LLM;
receive LLM responses from the LLM for each of the plurality of responses;
analyze the LLM responses with respect to a scoring rubric; and
generate a detail report defining performance of the medical provider during interaction between the patient and the medical provider.

10. The system of claim 9, wherein the patient is a virtual patient, and the capture device is a component of a virtual training device.

11. The system of claim 9, wherein the LLM operating on a processing device, or a group of processing devices, of the evaluation device.

12. The system of claim 9, wherein the LLM is executed remotely from the evaluation device.

13. The system of claim 9, the plurality of interview analysis variables each defining analysis parameters for the LLM.

14. The system of claim 9, the non-transitory executable instructions further comprising non-transitory executable instructions that, when executed by the processor operate to control the evaluation device to transmit one or more settings, respectively defined for each of the plurality of interview analysis variables, to the LLM to configure the LLM to analyze the segmented line in context with the transcribed text.

15. The system of claim 9, the non-transitory executable instructions further comprising non-transitory executable instructions that, when executed by the processor operate to control the evaluation device to transmit at least two of the prompts in parallel to at least two different instances of the LLM.

16. The system of claim 9, the non-transitory executable instructions further comprising non-transitory executable instructions that, when executed by the processor operate to control the evaluation device to generate a prompt for a binary analysis of one or more of the plurality of interview analysis variables.

17. The system of claim 9, the non-transitory executable instructions further comprising non-transitory executable instructions that, when executed by the processor operate to control the evaluation device to generate a prompt for a scaled analysis of one or more of the plurality of interview analysis variables.

18. The system of claim 17, the non-transitory executable instructions further comprising non-transitory executable instructions that, when executed by the processor operate to control the evaluation device to:
generate a first scaled-response prompt including the segmented line;
generate a second scaled-response prompt associated with the first scaled-response prompt querying the LLM to provide an example of a low-scoring end of the scale;
generate a third scaled-response prompt associated with the first and second scaled-response prompts querying the LLM to provide an example of a high-scoring end of the scale; and
generate a fourth scaled-response prompt associated with the first, second, and third scaled-response prompts querying the LLM to provide a rating on a given scale.

19. The system of claim 18, wherein the rating is a cosine similarity analysis between LLM responses associated with the first, second, and third scaled-response prompts.

20. A method for artificially intelligent debriefing dialog evaluation, comprising:
receiving captured data of a debriefing dialog for a scenario-based medical simulation;
transcribing audio of the captured data into transcribed text;
segmenting the transcribed text into segmented lines according to a speaker of the transcribed text within the audio;
generating a plurality of prompts, each prompt corresponding to one of a plurality of interview analysis variables, to control a large language model (LLM) to analyze each of the segmented lines in context of the transcribed text;
transmitting the plurality of prompts to the LLM;
receiving LLM responses from the LLM for each of the plurality of prompts;
analyzing the LLM responses with respect to a scoring rubric; and
generating a detail report defining performance of a trainee demonstrating scenario-based medical simulation based on the debriefing dialog.
